(19) United States
(12) Patent Application Publication
(10) Pub. No.: US 2010/0198271 A1
(43) Pub. Date: Aug. 5, 2010

(54) Screw Sheath for Minimally Invasive Spinal Surgery and Method Relating Thereto

(76) Inventor: Vincent Leone, Manhasset, NY (US)

Correspondence Address: GORDON & JACOBSON, P.C., 60 LONG RIDGE ROAD, SUITE 407, STAMFORD, CT, 06902, US

(21) Appl. No.: 12/364,368

(22) Filed: Feb. 2, 2009

(51) Int. Cl. A61B 17/58

(52) U.S. Cl. 606/302; 606/301

(57) ABSTRACT

A screw sheath is provided for advancing a pedicle screw from outside a patient to the vertebra during minimally invasive spinal surgery. The sheath includes an elongate tubular construct having an open proximal end, a tapered distal tip with an open end. The conical tip can be deformed or reconfigured when subject to the axial force of the pedicle screw being advanced into the pedicle or by manual operation of the operator. The pedicle screw is advanced through the sheath to the pedicle and retained by the tip of the sheath. Then a driver is used to advance the screw through the sheath and into the vertebra.

### BACKGROUND OF THE INVENTION

1. Field of the Invention

This invention relates broadly to surgical devices and methods. More particularly, this invention relates to devices for minimally invasive procedures on the spine and methods for minimally invasive spinal procedures.

2. State of the Art

Traditional open surgery on the spine requires a large incision and retraction of the tissue down to the vertebral surface. As a result of such large incisions the patient must suffer a relatively long recovery period as well as the greater potential for infection that is inherent with any open surgical procedure.

More recently, methods and systems have been developed for minimally invasive access to the spine. Minimally invasive spinal procedures use specialized instruments and implants in a through-port procedure which minimizes both recovery time and susceptibility to infection.

In a common minimally invasive spinal procedure for the implantation of pedicle screws on the vertebrae, a process of sequential dilation is used to provide access to the surgical site. In the process of sequential dilation, a needle cannula is first inserted under fluoroscopy through the tissue of the patient down to a subject vertebra. A guide wire is inserted through the needle cannula to the vertebral surface and impacted into the vertebra. The trajectory of the guidewire will correspond to the implanted pedicle screw. Therefore, the trajectory of the needle cannula and guidewire is reviewed by fluoroscopic examination, and removed and adjusted if necessary. Once the trajectory of the guidewire is confirmed, the needle cannula is removed and then a dilator is advanced over the guidewire down to the vertebral surface. Additional dilators are advanced in sequence over each other to progressively define an expanded working port through the tissue down to the surgical bed. For example, US Pub. No. 20060004398 to Binder, Jr. discloses a sequential dilation system, which is hereby incorporated by reference. Once the tissue has been expanded to a sufficient diameter for surgical access, the inner dilators are removed, leaving the outermost dilator as the working port through which the procedure can take place. A cannulated pedicle screw is then advanced over the guidewire and through the working port and implanted into the vertebra. After the pedicle screw is fully inserted, the guidewire is removed. The working port is preferably removed at the conclusion of the procedure, as additional components may be passed through the port to the surgical site even after seating of the pedicle screw.

Pedicle screws can be either monaxial or polyaxial. Polyaxial screws have multiple components coupled together such that the shaft of the screw is movable relative to a member that receives a engages a spinal rod for post-implantation adjustment. Both monaxial and polyaxial cannulated pedicle screws are often larger than their non-cannulated counterparts used in open surgery in order to accommodate a central bore required for advancement over the guidewire. In addition, a cannulated polyaxial pedicle screw is generally of more complex design than a non-cannulated one.

### SUMMARY OF THE INVENTION

According to one embodiment of the invention, a disposable sheath is provided for advancing a pedicle screw having a head and shaft, the pedicle screw being advanced from outside the patient to the surgical bed through the sheath in minimally invasive spinal surgery. The sheath includes an elongate tubular construct preferably having a cylindrical body with an open proximal end, a distal tip with a preferably open end, a relatively smooth interior so as to preferably not inhibit sliding advancement of the pedicle screw through the sheath, and a length sufficient to extend from outside the patient to the surgical bed but not so long as to be unwieldy during a procedure and interfere with radiographic visualization. The distal tip is preferably internally tapered to direct the distal end of the shaft of the pedicle screw toward the distal tip. The distal tip also includes a small diameter end adapted to be stabilized relative to a hole in the surgical bed thus maintaining appropriate delivery of the pedicle screw into the prepared vertebrae. The cylindrical body is sized to receive the entirety of the pedicle screw through the open proximal end and to allow the pedicle screw to be slidably advanced therethrough. The internally tapered tip retains the screw to prevent it from inadvertently passing completely through the sheath. The open end at the distal tip is sized to permit the distal end of the shaft of the pedicle screw to protrude therefrom. When the distal end of the shaft of the pedicle screw is properly aligned relative to the vertebra, the pedicle screw can be driven through the distal tip of the sheath, with the sheath capable of permanently or temporarily altering its distal diameter to permit the head of the pedicle screw to pass therethrough and into the vertebra.

In a preferred embodiment, the distal tip of the screw sheath has a conical shape, e.g., similar to a funnel with the larger portion of the funnel in adjacent the distal end of the proximal tubular body. The conical tip is preferably provided with radially oriented perforations or other defined structural weaknesses. The defined structural weaknesses are designed to controllably break the tip into a plurality of separate elements when subject to an axial force of sufficient magnitude of the pedicle screw being advanced into the vertebra. The proximal ends of the elements of the distal tip are preferably coupled to the distal end of the tubular construct by living hinge portions. In this embodiment, the sheath is a permanently alterable, disposable, single-use device and preferably made from paper, cardboard, or plastic.

In another embodiment of the invention, the internally tapered end of the screw sheath is comprised of multiple elements that are arranged radially inward into a first configuration, but may be moved radially outward into a second configuration. The elements may be biased into the first configuration. In the first configuration, the elements define a small opening at the distal end of the sheath sized to permit protrusion of the distal end of the shaft of the pedicle screw. In the second configuration, the elements radially expend relative to the longitudinal axis of the sheath so as to provide a distal opening of sufficient dimension for passage of the head of the pedicle screw. In this embodiment, the sheath is permanently or temporarily alterable, and may be a disposable or reusable device. The sheath may be made from any suitable material, including plastic and metal.

Also, according to the invention, a method is provided for advancing a pedicle screw to the surgical bed in a minimally invasive spinal surgical procedure. In the method of the invention, a needle cannula is introduced into the patient down to the surgical bed at the pedicle; i.e., at the desired location of screw insertion. A guide wire is inserted through the needle cannula to the pedicle and impacted into the vertebra. The trajectory of the guidewire is reviewed by fluoroscopic examination and the guidewire is removed and adjusted if necessary. Once the trajectory of the guidewire is confirmed, the needle cannula is removed and then a dilator is advanced over the guidewire down to the vertebral surface. Additional dilators, each of a progressively larger diameter, are advanced in sequence over each other to expand a space within the tissue down to the surgical bed. Once the tissue has been expanded to near a sufficient diameter for surgical access, a final working port is provided over the dilators, and the inner dilators are removed, leaving the working port through which the procedure can take place. An awl is advanced over the guidewire to enlarge the pilot hole formed by the guidewire in the pedicle, then removed, and then a tap is advanced to tap threads in the pilot hole for facilitating subsequent advancement of the pedicle screw. After the tap is removed, the guidewire is also removed to define an open space within the working port. The screw sheath is inserted into the working port, with the small diameter distal end of the screw sheath stabilized relative to and preferably within the pilot hole in the pedicle.

A preferably non-cannulated pedicle screw is inserted through the screw sheath to the pedicle, e.g., by slidably advancing the pedicle screw down through the bore of the screw sheath until the end of the distal shaft of the pedicle screw is guided into the pilot hole in the pedicle. Alternatively, the pedicle screw can be advanced down the screw sheath to be pre-positioned in the screw sheath prior to insertion of the screw sheath into the patient. A driver is then inserted into the screw sheath, engaged to the pedicle screw, and operated to rotationally advance the pedicle screw through the screw sheath and into the pedicle at the trajectory previously defined by the guidewire, awl and tap. As the pedicle screw is advanced, the distal tip of the screw sheath is opened, e.g., by the force of the advancing pedicle screw, separating the unitary tip into discrete elements expandable relative to each other or by movement of previously discrete elements relative to each other, to provide clearance for the shaft and the head of the pedicle screw to be advanced through the sheath and into the pedicle. Once the pedicle screw is stable in the pedicle, the screw sheath may be removed and, if necessary, the pedicle screw can be further advanced for additional purchase in the vertebra.

Using the screw sheath of the invention, several advantages are provided. As the pedicle screw does not need to be advanced over a guidewire to the surgical bed, a non-cannulated pedicle screw can be used. Therefore, any non-cannulated pedicle screw used in an open surgical procedure can be used in the procedure, enabling fewer components to support both open and minimally invasive systems. In addition, non-cannulated pedicle screws can be made smaller as they do not need to accommodate a bore for advancement over a guidewire. As such, using such non-cannulated pedicle screws in a minimally invasive procedures permits the resulting implants to be smaller in profile. This results in decreased tissue irritation and patient discomfort. Further, because the guidewire is removed prior to screw insertion, any opportunity for the guidewire to penetrate through the vertebral body and into the tissue beyond, which can occur if the guidewire inadvertently binds to the screw as the screw is advanced into the bone, is eliminated.

Additional objects and advantages of the invention will become apparent to those skilled in the art upon reference to the detailed description taken in conjunction with the provided figures.

### BRIEF DESCRIPTION OF THE DRAWINGS

FIG. 1 is a broken side elevation of a first embodiment of screw sheath according to the invention, shown in an original configuration.

FIG. 2 is a distal end view of the screw sheath of FIG. 1.

FIG. 3 is a view similar to FIG. 1, shown with the distal tip separated and expanded upon advancement of a pedicle screw.

FIG. 4 is a distal end of view of a second embodiment of a screw sheath with the distal tip in a contracted first configuration.

FIG. 5 is a broken side elevation view of the second embodiment of a screw sheath in a contracted first configuration.

FIG. 6 is a distal end of view of the second embodiment of a screw sheath with the distal tip in a dilated second configuration.

FIG. 7 is a broken view of a distal portion of a third embodiment of a screw sheath in a contracted first configuration.

FIG. 8 is a distal end view of the screw sheath of FIG. 6 in the first configuration.

FIG. 9 is a broken view of the distal portion of the third embodiment of the screw sheath in an expanded second configuration.

FIG. 10 is the distal end view of the screw sheath of FIG. 9 in the second configuration.

FIG. 11 illustrates a needle cannula and a guidewire inserted into a patient.

FIG. 12 illustrates a suitable trajectory for the guidewire.

FIG. 13 illustrates a tissue dilator inserted over the guidewire and down to the surgical bed.

FIG. 14 illustrates progressive dilation of the tissue down to the surgical bed.

FIG. 15 illustrates tapping a pilot hole about the guidewire.

FIG. 16 illustrates a screw sheath inserted through the working port.

FIG. 17 illustrates a pedicle screw inserted through the screw sheath.

FIG. 18 illustrates a pedicle screw advanced through the distal end of the screw sheath and into the vertebra.

FIG. 19 illustrates the pedicle screw fully implanted at the vertebra.

### DETAILED DESCRIPTION OF THE PREFERRED EMBODIMENTS

Turning now to FIG. 1, a screw sheath 10 for minimally invasive spinal surgery according to an embodiment of the invention is shown. The screw sheath includes an elongate cylindrically tubular body 12 defining a proximal end 14, a distal end 16 and an axial bore 18 with a preferably smooth interior surface extending between the proximal and distal ends. The tubular body 12 preferably has a length of 15-25 cm, a bore 18 diameter of 15-22 mm, and a wall thickness of, e.g., 0.5-2 mm. The length is sufficient to extend from outside the patient to the surgical bed at the vertebral body but not so long as to be unwieldy during a procedure. The diameter is suitable to pass pedicle screws. The wall thickness is material dependent, but of sufficient thickness to have a rigidity to support the tubular body without buckling under normal use.

The proximal end 14 may have a flared opening 20. The distal end 16 preferably has a conically tapering tip 22 with a distal opening 24 having a crosswise dimension smaller than the diameter of the bore 18. The tip 22 is preferably both internally and externally tapered. The flared opening 20 facilitates receiving a pedicle screw 30 into the bore 18, the smooth interior of the bore 18 is designed to not inhibit sliding advancement of the pedicle screw through the sheath, the tip 22 is externally tapered to facilitate stabilize the sheath 10 relative to a hole in the bone, and the tip is internally tapered to guide the distal end of the pedicle screw toward the hole in the bone, all described further below. The tapered tip 22 is sized to retain the pedicle screw 30 to prevent it from inadvertently passing through the distal end 16 of the sheath 10, but to permit the distal shaft 34 of the screw to preferably slightly protrude therefrom.

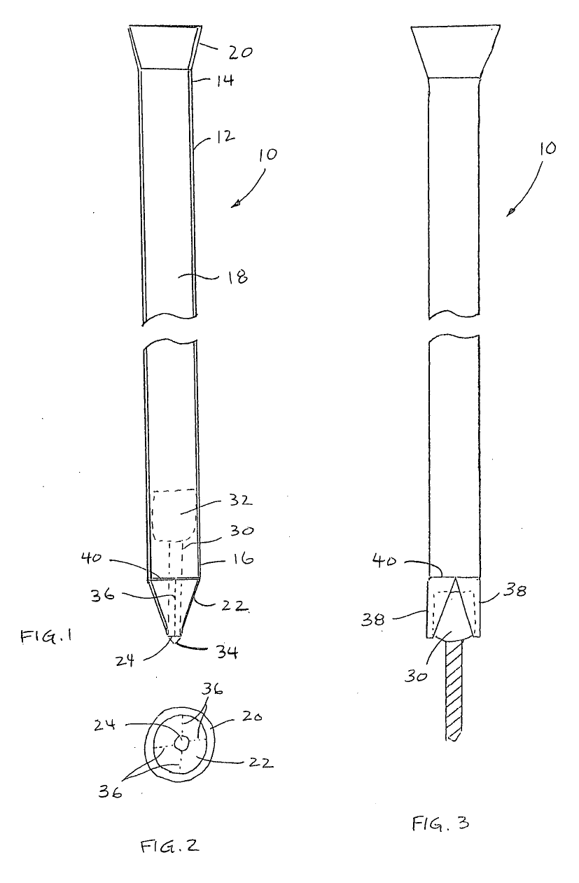

As shown in FIGS. 2 and 3, the tip 22 is preferably provided with several sets of radially oriented perforations 26. Referring to FIG. 3, the perforations 26 are designed to controllable break in at least a longitudinal direction when subject to an axial force under conditions of engagement between the distal end of the shaft of the pedicle screw and the bone and rotational advancement of the screw relative to the sheath and into the bone, as discussed in more detail below. Under such force the tip separates into a plurality of separate elements 38 attached with living hinges 40 to the distal end 16 of the tubular construct 12. Referring to FIG. 3, once the tip 22 separates along the perforations 36, the tip 22 is openable to a diameter suitable to permit passage of the pedicle screw 30 completely therethrough, and preferably at least the same diameter as the bore 18. In this embodiment, the screw sheath 10 is a permanently alterable, disposable, single-use device and preferably made from paper, cardboard or plastic.

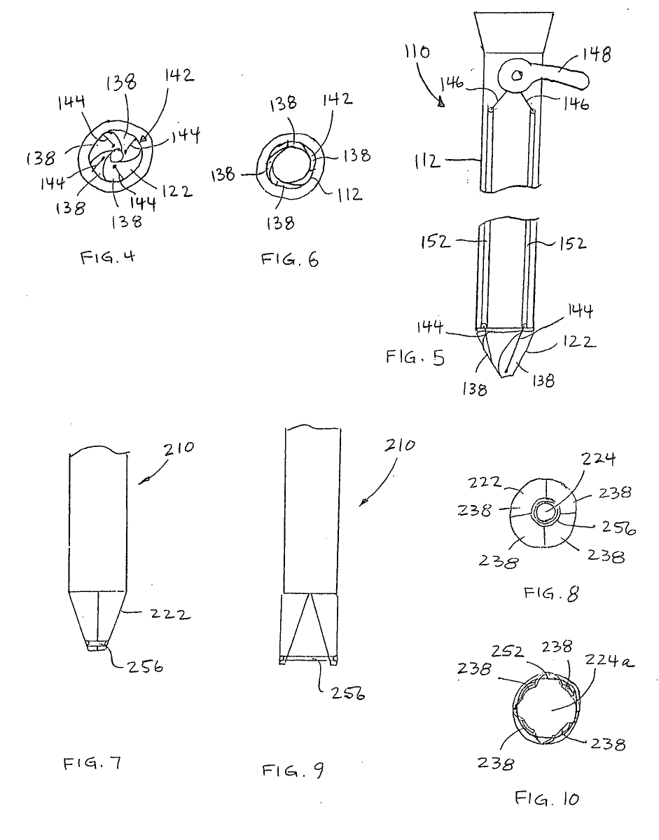

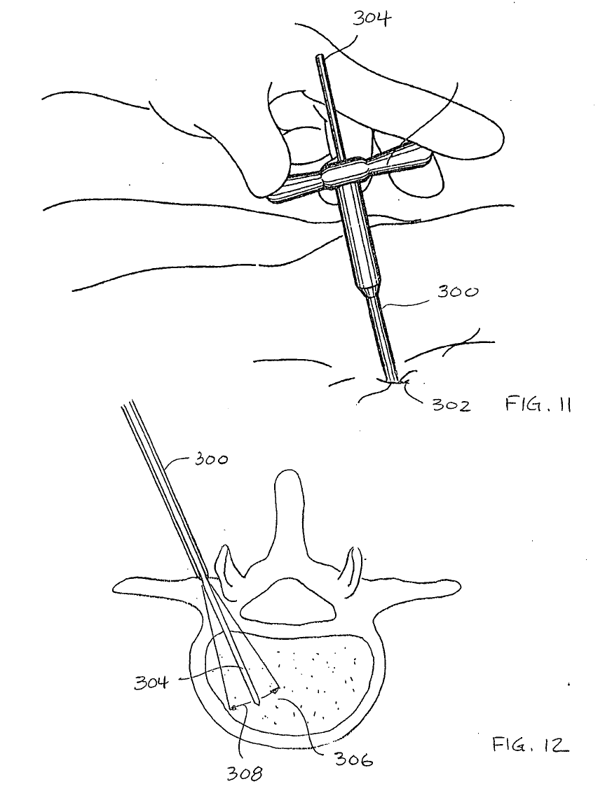

Turning now to FIGS. 4 and 5, a second embodiment of a screw sheath 110 generally similar to the first embodiment (with like parts having reference numerals incremented by 100) is shown. The distal tip 122 of the screw sheath includes a plurality of elements 138 forming an expandable conically-shaped iris 142 biased toward the closed position. The bias may be imparted by the materials and molded structure of sheath 110, and/or by springs or elastic elements coupled to the iris elements. The iris elements 138 are rotatable counter to the bias and relative to the axis of the tubular body 112 to open the iris 142 from a first diameter (FIGS. 4 and 5) sized to permit only the end of the pedicle screw shaft to protrude to a second diameter (FIG. 6) permitting passage of the entirety pedicle screw therethrough. The elements 138 of the iris 142 can be reconfigured from the first to the second diameters with one or more control wires 144 coupled to one or more of the iris elements 138 and retraction of such control wires longitudinally relative to the tubular body 112. Referring to FIGS. 4 through 6, in order to retract control wires 144, the proximal ends 146 of the control wires 144 are coupled to a lever 148, knob or other handle element operable by the user such that when operated, movement of the distal ends of the control elements results. The control wires 144 may extend within lumen 152 defined within or along the wall of the tubular body 112. Alternatively, the elements 138 can be adapted to automatically open upon rotation of the pedicle screw through the tip 122. In these embodiments, the screw sheath 110 is either temporarily or permanently alterable, may be disposable or re-usable and may be made from plastic, metal or other suitable materials.

Referring to FIGS. 7 and 8, a screw sheath 210 generally similar to the first embodiment (with like parts having reference numerals incremented by 200) is shown. The distal tip 222 of the screw sheath includes a plurality of discrete elements 238 defining a conical shape with an open end 224. The discrete elements 238 are coupled together with an elastic band 256 that biases the elements into the conical configuration, permitting only the distal shaft of a pedicle screw to protrude therefrom. Referring to FIGS. 9 and 10, as a pedicle screw is forcibly advanced through the opening 224 in the distal tip 222, the pedicle screw acts on the distal tip to moved the discrete elements 238 against the bias of the band 256 and to open the tip 222 to define a larger space 224a to permit passage of the pedicle screw. The sheath 210 is preferably temporarily alterable, with the tip returnable to a conical shape 222 after passage of a pedicle screw, and may be disposable or re-usable and may be made from any suitable materials.

Using any of the embodiments of the screw sheath described above, a minimally invasive surgical procedure on the spine is now described. Initially, the skin of the patient is punctured and an incision through the underlying tissue is made and enlarged so that surgical instruments can be worked and implants, including pedicle screws, can be implanted along the surgical bed of one or more pedicles of the vertebrae of the spine in accord with the surgical procedure. For example, referring to FIGS. 11 and 12, a needle cannula 300 is used to create an incision 302 to provide access to the posterior spine. After the needle cannula 300 is inserted, a 2 mm guidewire 304 is fed through the cannula 300 and impacted into the vertebrae 306. The trajectory of the guidewire 304 is examined under fluoroscopy and confirmed to be within suitable limits 308. If the trajectory is improper, the guidewire 304 is removed and reinserted at an acceptable trajectory. After the trajectory of the guidewire 304 is approved by the surgeon, the needle cannula 300 is removed from over the guidewire.

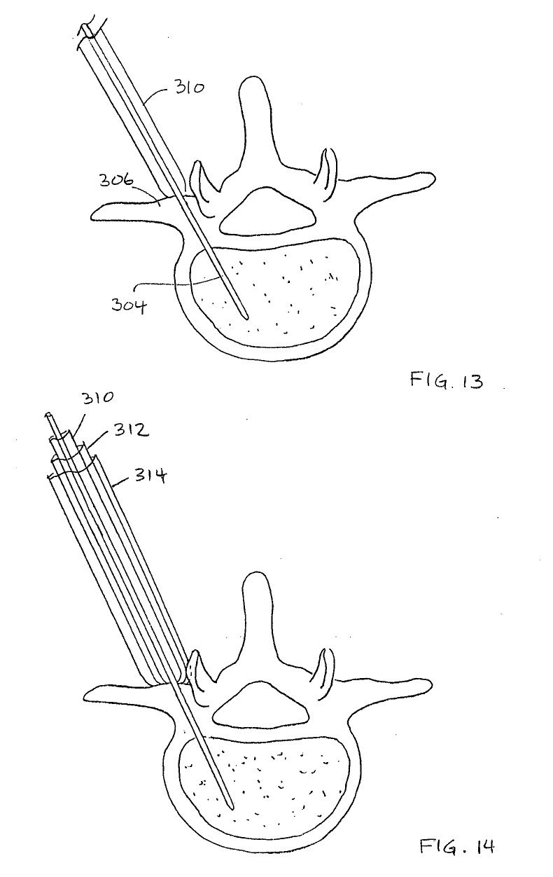

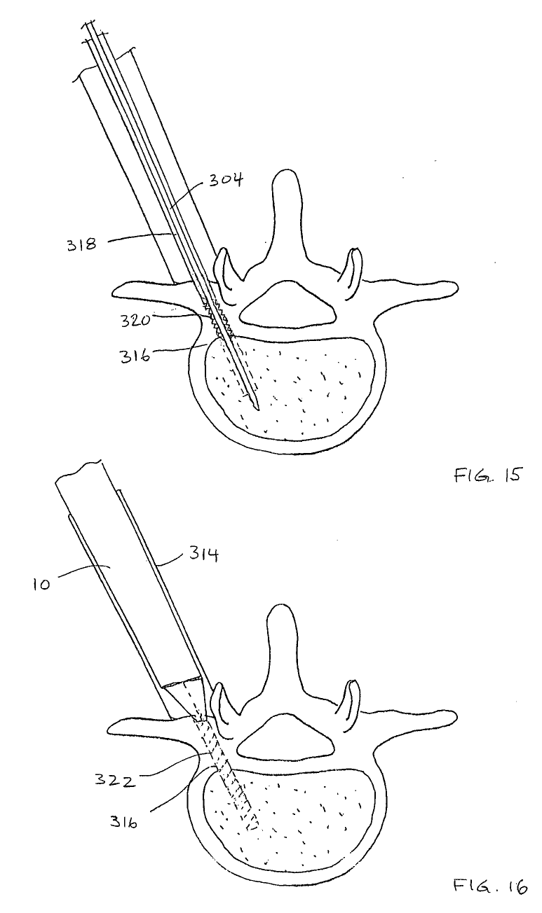

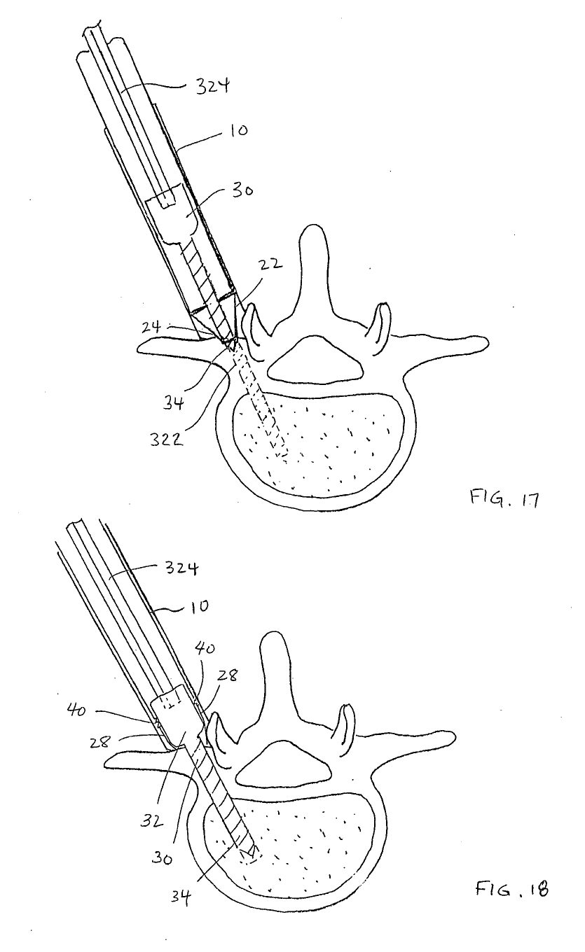

Turning to FIG. 13, a dilator 310 (e.g., 5 mm diameter) is advanced over the guidewire 304 down to the surgical bed on the vertebra 306, operating to separate muscle and other tissue from about the guidewire 304 to expand a working space. As shown in FIG. 14, additional dilators, e.g. 312, 314, each of a progressively larger diameter (e.g., 7 mm and 9 mm), are advanced in sequence over each other to further define a larger working space within the tissue down to the surgical bed. Additional intermediate or larger dilators may be used. The tissue is expanded by the dilators to a size able to accommodate the outer diameter of a screw sheath 10, 110, 210, described above. Once the tissue is sufficiently expanded, the inner dilators 310, 312 are removed, leaving only the outermost dilator 314 in place as a working port to prevent movement of the tissue over the working area.

A cannulated awl is then advanced within the working port 314 over the guidewire 306 to define a pilot hole 316 about the guidewire 304 in the pedicle. After the pilot hole is formed, the awl is removed. Then the distal end 320 of a tap 318 is advanced to form threads 322 (FIG. 15) in the pilot hole 316 to facilitate subsequent advancement of the pedicle screw into the hole. Then the tap 318 is removed. The guidewire 304 is also removed. This provides a degree of safety by eliminating the potential for harm possible when a guidewire is left within the vertebra and a cannulated pedicle screw is advanced thereover, as is done in prior art minimally invasive systems. For example, when the guidewire is staged within the vertebra and the cannulated pedicle screw is advanced thereover, the opportunity exists for the pedicle screw to bind to the guidewire and move the guidewire through the vertebra as the pedicle screw is thread into the vertebra. This can inadvertently cause the guidewire to penetrate through the far side of the vertebra and thereafter puncture tissue and organs causing significant patient harm. Removing the guidewire eliminates this potential harm. Once the guidewire is removed, an at least partially threaded bore 322 (FIG. 16) is provided in the pedicle for insertion and engagement with the pedicle screw.

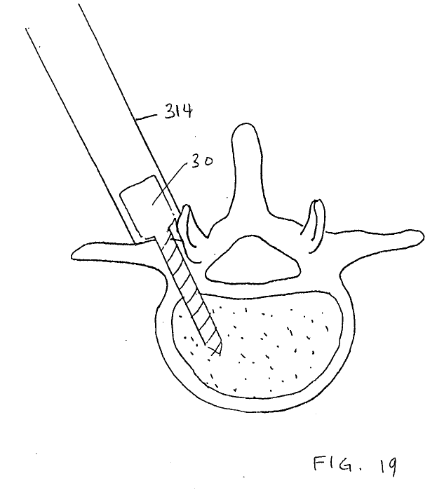

Referring to FIG. 16, the screw sheath (for convenience referred to as 10, but intended to refer to any screw sheath) is then provided into the working port 314 with the distal tip 22 of the sheath manually stabilized relative to the entry of the pilot hole 322 in the surgical bed. As shown in FIG. 17, a preferably non-cannulated pedicle screw 30 is provided. The pedicle screw is either a monaxial or polyaxial screw, such as the type used in open-surgery for the same type of spinal correction. The screw 30 is inserted into the bore 18 of the screw sheath 10 down to the surgical bed, e.g., by slidably advancing the pedicle screw down through the bore 18 of the screw sheath 10 until the distal shaft 34 of the pedicle screw protrudes from the distal opening of the tip 22 of the screw sheath and into the threaded bore 322 in the pedicle. Alternatively, the pedicle screw can be slid down the screw sheath to be pre-positioned in the distal end of the screw sheath prior to insertion of the screw sheath into the patient. As the pedicle screw does not need to be advanced over a guidewire to the surgical bed, a non-cannulated pedicle screw can be used. As such, the smaller, less costly to manufacture, non-cannulated pedicle screws used in open surgical procedures can be used in this minimally invasive procedure. Thus, the screw sheath 10 enables the use of a common pedicle screw to support both open and minimally invasive systems and procedures.

A driver 324 is then inserted into the screw sheath, engaged to the proximal end of the pedicle screw 30, and operated to rotationally drive the pedicle screw. This causes the threads on the shaft at the end of the pedicle screw to engage relative to the threaded hole in the bone and draws the pedicle screw into the bone at the trajectory defined by the guidewire, awl and tap. Referring to FIGS. 17 and 18, as the pedicle screw 30 is advanced by the driver 324 into the threaded hole 322, the distal tip of the screw sheath 10 is opened, with the tip separating about perforations 36 (FIG. 2) into discrete elements 28 that deform about living hinges 40. While one description of opening the distal tip is described, the distal tip may be opened in accord with any suitable mechanism, including any of the mechanisms described above; i.e., by separation of attached portions of the tip into multiple discrete elements or by movement of previously discrete elements relative to each other, so as to provide clearance for the entirety of the shaft 34 and the head 32 of the pedicle screw 30 to be advanced through the sheath and into the pedicle.

Once the pedicle screw is stable in the pedicle, which may occur either before or after the pedicle screw 30 is fully seated at the surgical bed, the screw sheath 10 can be removed, as shown at FIG. 19. If the screw sheath is removed prior to the pedicle screw being fully seated, the pedicle screw 30 is advanced as necessary with the driver until it has suitable purchase in the vertebra. If the screw sheath is of a disposable design, the sheath is discarded, and the procedure may be repeated with another screw sheath for additional pedicle screws at other locations. If the screw sheath is of a re-usable design, the sheath can be cleared of any debris and be re-used for seating additional pedicle screws during the procedure. Additionally, such re-usable screw sheaths may be re-sterilized for re-use in subsequent surgical procedures.

The working port 314 is removed at the conclusion of implantation of the pedicle screw and any procedure requiring access through the working port.

There have been described and illustrated herein several embodiments of a screw sheath for a minimally invasive spinal surgery and methods of implanting a pedicle screw using a screw sheath. While particular embodiments of the invention have been described, it is not intended that the invention be limited thereto, as it is intended that the invention be as broad in scope as the art will allow and that the specification be read likewise. For example, while the screw sheath permits use of a non-cannulated screws in a minimally invasive manner, it is appreciated that cannulated screws can likewise be used within the sheath and in accord with the method described. It will therefore be appreciated by those skilled in the art that yet other modifications could be made to the provided invention without deviating from its spirit and scope as claimed.

What is claimed is:

1. A screw sheath for minimally invasive spinal surgery for advancing a pedicle screw from outside a patient to a vertebra of the patient, the pedicle screw having a head and shaft with a distal point, the screw sheath comprising:
a) an elongate tubular body having a proximal end, a distal end, a length of 15 to 25 cm between the proximal and distal ends, and an axial bore sized to receive the pedicle screw;
b) a tip at said distal end that is externally tapered and provided with an opening of a first dimension sized to permit the distal point of the pedicle screw to protrude but retain the head of the pedicle screw, said tip of said sheath including a predefined weakness along which said tip is intended to break apart when subject to an axial force of a magnitude required to advance the pedicle screw relative to the screw sheath and into the vertebra.

2. A screw sheath according to claim 1, wherein:
said tip is internally tapered.

3. A screw sheath according to claim 1, wherein:
said tip is conical in shape.

4. A screw sheath according to claim 1, wherein:
said tip is non-metallic.

5. A screw sheath according to claim 1, wherein:
said axial bore has a diameter of 15-22 mm.

6. A screw sheath according to claim 1, wherein:
said tubular body includes a smooth interior surface that defines said bore.

7. A screw sheath for minimally invasive spinal surgery for advancing a pedicle screw from outside a patient to a vertebra of the patient, the pedicle screw having a head and shaft with a distal point, the screw sheath comprising:
a) an elongate tubular body having a proximal end, a distal end, a length of 15 to 25 cm between the proximal and distal ends, and an axial bore having a diameter sized to receive the pedicle screw; and
b) a tip at said distal end having an opening in a first configuration with a first cross-wise dimension smaller than said diameter of said bore so as to prevent passage of the pedicle screw, said tip being reconfigured into a second configuration with a second cross-wise dimension sized to permit passage of the pedicle screw.

8. A screw sheath according to claim 7, wherein:
said tip includes perforations, wherein in said first configuration said tip is intact about said perforations and in said second configuration said tip separates along said perforations to define elements that radially displace relative to each other.

9. A screw sheath according to claim 7, wherein:
said tip defines discrete elements that are biased into said first configuration, and can be moved against bias into said second configuration.

10. A screw sheath according to claim 7, wherein:
said tip is tapered.

11. A screw sheath according to claim 10, wherein:
said tip is tapered internally.

12. A screw sheath according to claim 10, wherein:
said tip is tapered externally.

13. A screw sheath according to claim 10, wherein:
said tip is conical.

14. A kit for minimally invasive spinal surgery, comprising:
a) a pedicle screw having a head with a recess for a driver, and shaft for insertion into bone, the shaft having a shaft end opposite said recess; and
b) an elongate tubular body having a proximal end, a distal end, a length of 15 to 25 cm between said proximal and distal ends, an axial bore having a diameter sized to receive said pedicle screw, and a tip at said distal end having an opening in a first configuration with a first cross-wise dimension smaller than said diameter of said bore so as to prevent passage of said pedicle screw, said tip being reconfigured into a second configuration with a second cross-wise dimension sized to permit passage of said pedicle screw.

15. A kit according to claim 14, wherein:
said pedicle screw is non-cannulated.

16. A kit according to claim 14, wherein:
said pedicle screw is polyaxial.

17. A kit according to claim 14, wherein:
said pedicle screw is monaxial.

18. A kit according to claim 14, further comprising:
a plurality of pedicle screws and sheaths.

19. A kit according to claim 14, wherein:
said tip includes perforations, wherein in said first configuration said tip is intact about said perforations and in said second configuration said tip separates along said perforations to define elements that radially displace relative to each other.

20. A kit according to claim 14, wherein:
said tip is tapered.

21. A kit according to claim 14, wherein:
said tubular body of said sheath is made of one of paper, cardboard and plastic.

22. A method of implanting a non-cannulated pedicle screw in a vertebra of a patient, comprising:
a) dilating tissue of the patient down to the vertebra;
b) inserting a screw sheath within the dilated tissue, the screw sheath having an elongate tubular body having a proximal end, a distal end, a length of sufficient to extend from outside the patient down to the vertebra, an axial bore sized to receive the pedicle screw, and a tip at said distal end having an opening sized to retain the pedicle screw;
c) first advancing a non-cannulated pedicle screw through the sheath to the tip so that the pedicle screw is retained by the tip;
d) after said first advancing, second advancing the non-cannulated pedicle screw completely through the tip and into the vertebra; and
e) removing said screw sheath from the patient.

23. A method according to claim 22, wherein:
said second advancing includes reconfiguring said tip.

24. A method according to claim 23, wherein:
said reconfiguring said tip includes separating attached portions of said tip into multiple portions movable relative to each other.

25. A method according to claim 24, wherein:
said separating includes separating about perforations.

26. A method according to claim 23, wherein:
said reconfiguring said tip includes moving discrete elements relative to each other so as to permit passage of the entirety of the pedicle screw.

27. A method according to claim 22, further comprising:
prior to dilating the tissue of the patient, inserting a guidewire into patient, wherein said dilating the tissue is performed over the guidewire; and
removing the guidewire prior to inserting the screw sheath.

28. A method according to claim 27, wherein:
said dilating includes progressively dilating the tissue with dilators of different diameter.

29. A method according to claim 27, further comprising:
after said dilating and prior to removing the guidewire, a pilot hole is formed in the vertebra coaxial with the guidewire.

30. A method according to claim 22, wherein:
said second advancing includes rotationally driving the pedicle screw.

31. A method according to claim 22, wherein:
said tip of said screw sheath is manually retained relative to said pilot hole during said second advancing.

32. A method according to claim 31, wherein:
said tip of said screw sheath is manually retained relative to said pilot hole during said first advancing.
